(19) United States
(12) Patent Application Publication
(10) Pub. No.: US 2006/0148027 A1
(43) Pub. Date: Jul. 6, 2006

(54) Sprouty and SPRED protein biosensors

(75) Inventors: Steven L. McKnight, Dallas, TX (US); Xinle Wu, Dallas, TX (US); Pia Vogel, Dallas, TX (US); Peter Alexander, Dallas, TX (US); Julian Bill Peterson, Dallas, TX (US)

Correspondence Address: RICHARD ARON OSMAN;SCIENCE AND TECHNOLOGY LAW GROUP, 242 AVE VISTA DEL OCEANO, SAN CLEMEMTE, CA, 92672, US

(73) Assignees: Board of Regents, the University of Texas System; Southern Methodist University

(21) Appl. No.: 11/327,834

(22) Filed: Jan. 5, 2006

Related U.S. Application Data
(63) Continuation of application No. 60/642,249, filed on Jan. 6, 2005.

(51) Int. Cl. C12Q 1/26

(52) U.S. Cl. 435/25

(57) ABSTRACT

Sprouty/SPRED protein cysteine-rich domain (SCRD) modules comprising an iron:sulfer complex are used to sense electro/chemical signals.

### CROSS-REFERENCE

This application is a continuation of 60/642,249 filed Jan. 6, 2004.

### GOVERNMENT RIGHTS

This work was supported by NIM Grant 5R37MH05938807. The U.S. government may have rights in any patent issuing on this application.

### FIELD OF THE INVENTION

The field of the invention is electro/chemical devices formed with Sprouty and SPRED protein modules and complexes.

### BACKGROUND OF THE INVENTION

The Sprouty and related SPRED family of proteins are negative regulators of a number of intracellular signaling pathways in a variety of metazoan animals; e.g. Kim & Bar-Sagi, 2004, Nature Reviews 5, 441-450; Goodman, US Pat Pub 20040091895; Nonami et al. Genes Cells. September 2005;10(9):887-95; Sasaki et al. Nat Cell Biol. May 2003;5(5):427-32;Kato et al. BBRC Mar. 21, 2003;302(4):767-72; Lim et al. Mol Cell Biol. November 2002;22(22):7953-66; Wakioka et al. Nature. Aug. 9, 2001;412(6847):647-51; Lim et al. J Biol Chem. Oct. 20, 2000;275(42):32837-45.

For example, the respective genomes of mice and humans encode three highly related Sprouty proteins, designated Sprouty 1, Sprouty 2 and Sprouty 4. These two mammalian genomes likewise encode three related SPRED proteins designated SPRED1, SPRED2 and SPRED3. Furthermore, these proteins can exist in alternatively spliced forms; e.g. Wang et al. 2003, Intnl J Mol Med 12, 783-87.

The Sprouty and SPRED proteins are themselves related in primary amino acid sequence comprising a C-terminal region of approximately 120 amino acids (e.g. FIG. 1 Kim 2004, supra; FIG. 5 of Lim 2000, supra)—we term this domain or module the Sprouty/SPRED protein cysteine-rich domain (SCRD). When compared by standard methods of amino acid sequence alignment, these SCRD domains reveal highly significant arrangements of cysteine residues with three unusual features. First, the abundance of cysteine residues in these regions is considerably higher than the cysteine density in normal, intracellular proteins. Second, these conserved cysteine residues are grouped in an unusual manner in which they are often separated by only one or two other amino acids. Third, the pattern of cysteine residues is stereotypically conserved, not only within each of the two sub-families (the Sprouty family and the SPRED family), but also within the larger family composed of all six proteins.

### SUMMARY OF THE INVENTION

An artificial nano- or micro-electrical device comprising or consisting essentially of a SCRD module; such as wherein the device or the module operates as a micro- or nano-transistor, battery, sensor, circuit or switch; such as wherein the device operates as a nitric oxide sensor or nitric-oxide sensitive switch.

In particular embodiments, the device comprises or consists essentially of a first SCRD module electrically coupled to a partner module, wherein the respective redox potentials of the modules effect electron transport between the modules to form a circuit; such as wherein the partner module is a different, second SCRD module; such as wherein the device comprises or consists essentially of plurality of redox-linked SCRD modules forming a circuit; such as wherein the device is incorporated in an electronic micro- or nano-chip.

The invention also provides an assay for agents which modulate the interaction between a nano- or micro-electrical device comprising or consisting essentially of a SCRD module and a cellular component target, the assay comprising the steps of: (a) contacting a mixture comprising the device, the target, and a candidate agent under conditions wherein but for the presence of the agent, the device and target engage in a first interaction; and (b) detecting a second interaction between the device and target, wherein a difference between the first and second interactions indicates that the agent modulates the interaction between the device and target.

In particular embodiments, the agent modulates an electrical connection between the device and target; the agent insulates an electrical connection between the device and target; the target is selected from a polynucleotide and a protein; and the target is a protein of FGF signaling (involved in neurogenesis and CNS diseases like stroke), EGF signaling (involved in cancer), or VEGF signaling (involved in angiogenesis, ischemia and cancer).

The invention also provides a method for detecting nitric oxide comprising the step of contacting a nitric oxide sensor or switch (supra) with a reagent, wherein the sensor or switch indicates the-presence of nitric oxide.

### DETAILED DESCRIPTION OF SPECIFIC EMBODIMENTS OF THE INVENTION

Upon expression and purification of all members of both Sprouty and SPRED proteins under recombinant, inducible production from the bacterium, Escherichia coli, we discovered that the Sprouty and SPRED proteins exhibited an amber coloration. Such coloration is unusual for most proteins that, under visible light, tend to show no color. The fact that the coloration resembled that of rust, it was speculated that the Sprouty and SPRED proteins might contain oxidized iron. Such iron could come in the form of either heme, a standard, iron-containing prosthetic group found in a small subset of proteins from all kingdoms of life. Alternatively, the rust-like color might be attributed to the association of iron in the Sprouty and SPRED proteins of one of a small group of prosthetic groups designated “iron:sulfur” complexes. By use of both chemical probes of free sulfur and atomic adsorption spectroscopy, we discovered that the Sprouty and SPRED proteins are rust-colored due to the association of iron:sulfur complexes.

Iron:sulfur complexes have been described in a modest number of proteins that utilize these two elemental metals as parts of enzymatic mechanisms, or as prosthetic groups that allow a protein to act as a molecular sensor to either redox state or elemental gas. Iron:sulfur complexes can exist in either the oxidized state where the protein displays its rust-like color, the reduced state where the protein is colorless, or a gas-bound state which is again, typically, colorless. In the vast majority of cases, if an iron:sulfur complex is designed to act as a gas sensor, the relevant gas is nitric oxide. We found that when the Sprouty and SPRED proteins are produced in E. coli, they are colored, indicative of the fact that they come out of bacterial cells in the oxidized state. When subjected to dithionite, a strong chemical reducing agent, both Sprouty and SPRED proteins lose their color, reflective of the iron sulfur complex having been converted to the reduced state.

One of our hypotheses for the function of Sprouty was that it should be a sensor of the redox state of cells and could serve as a link between metabolic pathways and regulation of intracellular signaling. To do this, Sprouty would have to be able to “sense” the reducing potential of the cell and this could only be done if its redox potential was in a metabolically useful range—between −250 mV and −350 mV. In fact, the most useful range would be to reflect the potential of reduced pyridine nucleotides (NAD and NADP) whose redox potentials are approximately −320 mV. If our hypothesis was correct, Sprouty should have a redox potential around −300 mV. To measure the redox potential of Sprouty, we took advantage of Sprouty's color change going from colored to colorless upon reduction. We selected a small molecule redox dye (Safranine O, −289 mV) from a collection of such dyes for the following properties: 1) its color did not interfere with the change in color of Sprouty, 2) its redox potential was −289 mV, 3) it would accept electrons from a photochemical reductant-deazariboflavin [1]. A solution of Sprouty was dissolved in buffer with Safranine O and a catalytic concentration of 5-deazariboflavin and placed in a quartz cuvette under an atmosphere of argon. The absorbance spectrum of the sample was recorded. A high intensity white light was used to photo-reduce the Safranine O and Sprouty which were in redox equilibrium and the spectrum recorded. Using standard analytical techniques, we determined the redox potential of Sprouty to be −309 mV which is in the range for a redox sensor as we had proposed. Interestingly, SoxR, an oxidative stress response transcriptional activator protein in E. coli, also responds to the redox state of this bacterium in a similar fashion [2].

These initial discoveries have been confirmed and extended by a sophisticated method of spectroscopy designated electron paramagnetic resonance (EPR). We inspected the Sprouty 2 protein by EPR under three conditions, oxidized, reduced or bound to nitric oxide. Electron paramagnetic resonance (EPR) measurements were performed on Sprouty proteins using a Bruker model X-band. No EPR signals were observed of the oxidized form of Sprouty even at low temperature (100 K). Upon addition of the reducing agent dithionite to oxidized Sprouty protein, EPR signals were observed that are characteristic for iron:sulfur complex-containing proteins. Reviewing our EPR spectrum of Sprouty in the oxidized state and reduced state, the EPR signal with the characteristic g-value of 1.998 for reduced 2Fe-2S clusters is only seen in the spectrum of reduced Sprouty shown in red and is comparable to spectra of other 2Fe-2S clusters described in the literature [3]. In control experiments, dithionite was added to a solution containing the non-FeS protein, bovine serum albumin, no such spectra were observed.

To test the potential of Sprouty as a nitric oxide sensor, NO was added to Sprouty in solution. In subsequent EPR measurements of a mixture of Sprouty with NO, a distinct new signal with a g-value of 2.041 was apparent which matches exactly the EPR spectra of dinitrosyl iron complex signals described in literature [4]. In control experiments we again treated the non-FeS protein, bovine serum albumin, with NO. No EPR signals were observed under such conditions.

The apparent affinity of the Sprouty Fe-S cluster to NO was tested by NO-titration experiments. Our data show that binding of NO to reduced Sprouty is very tight. The apparent dissociation constant was estimated to be below micromolar NO concentration. The binding affinity of oxidized Sprouty for NO was clearly less pronounced, indicating a NO sensor activity that depends on the redox state of the Sprouty protein. NO binding has also been reported to the E. Coli iron-sulfer protein SoxR. The NO bound protein changes activity in a similar fashion [5].

In aggregate, chemical, biochemical and spectroscopic studies of the Sprouty and SPRED proteins demonstrate that these proteins bind iron: sulfur prosthetic groups for the purpose of forming dedicated microsensors of either redox state, nitric oxide, or both. In subsequent experiments we demonstrate that the SCRD module is sufficient for these functions: when expressed alone or recombined with a variety of fusion partners, SCRD domains retain their ability to form functional microsensors of either redox state, nitric oxide, or both. Functional association, such as by way of structural linkage (e.g. fusion), with other functional domains provide function dependence between the functional domains, which, in various embodiments, provide for electron transport circuits, functionally regulated switches and circuits, etc. For example, ligand-binding domain partners can provide redox sensitive ligand binding, or ligand-binding sensitive redox signaling.

In addition, we show that sequence variation across SCRD modules provide variation in redox potential, permitting electron transport. A large library of 5,000 SCRD modules subject to random, partially random, and directed mutagenesis is used to select for a metabolically useful redox potential range in mV and sub-mV (e.g. 0.1 mV) increments between −250 mV and −350 mV.

In further biochemical characterization of the Sprouty and SPRED proteins it was noted that the proteins might form large aggregates. When chromatographed over gel filtration columns typically used to resolve and separate proteins of normal size (10,000 to 250,000 daltons), the Sprouty and SPRED proteins were observed to elute at or close to the void volume. This chromatographic behavior provisionally indicated that the Sprouty and SPRED proteins might aggregate into multi-subunit complexes so large that they would be unable to enter the micropores of the gel filtration matrix. In order to definitively evaluate this observation a gel filtration column capable of separating very large protein complexes was utilized.

Sprouty was cleaved from maltose binding protein (MBP) using 0.1 mg/mL TEV protease at 4° C. overnight. Sprouty and MBP were separated using a Superose 6 10/300 GL column and their identities were confirmed by SDS-PAGE. While MBP eluted at a volume consistent with its monomeric molecular weight of 44 kDa, Sprouty still eluted very early, confirming the aggregation comes from one Sprouty protein and not MBP. Gel filtration provides an estimation of the complex size in terms of its Stokes radius. The elution volume of Sprouty corresponds to a Stokes radius of 110 angstroms. Similar studies of SPRED proteins confirmed that they also form large aggregates.

Three additional methods were employed to confirm that the Sprouty and SPRED proteins form large aggregates of biological relevance. First, after liberation from MBP via TEV proteolytic cleavage, the Sprouty protein was subjected to both velocity and equilibrium sedimentation in an analytical ultracentrifuge. Analytical ultracentrifugation experiments were performed using a Beckman XL-I analytical ultracentrifuge. Sedimentation velocity data were collected at 280 nm, 20° C., and 40,000 rpm and analyzed using the second moment method in the Beckman software. This analysis predicted a sedimentation of 37S for the Sprouty protein.

For sedimentation equilibrium experiments, samples were loaded in an An60Ti rotor and run at 4,000 and 7,000 rpm, at 4° C. Data were collected at a wavelength of 280 nm. Background absorbance was estimated by overspeeding at 42,000 rpm until a flat baseline was obtained. Analysis of the data, including estimation of molecular weight, was carried out using the Beckman software. This analysis resulted in a molecular weight prediction of 3.1 Mda, which corresponds to a complex consisting of roughly one hundred Sprouty monomers.

In addition to velocity and equilibrium sedimentation, both of which confirmed that the Sprouty protein forms a large, multi-subunit complex, we inspected the properties of the complex directly by electron microscopy. Imaging by negative staining revealed uniform, globular particles with a diameter of 140 angstroms.

Samples of purified Sprouty protein (5 μl at 0.1 mg/ml) were applied to carbon-coated copper grids and stained with 1% uranyl acetate. Samples were then viewed with the JEOL 1200 CX electron microscope at 80 kV.

Our results demonstrate that Sprouty and SPRED proteins can function as iron:sulfur-containing sensors and form large ordered aggregates. These data are consistent with numerous papers that have studied Sprouty and SPRED proteins by immunofluorescence in mammalian cells, and shown highly punctate staining patterns for the Sprouty and SPRED proteins [6, 7], yet mistakenly interpreted such staining patterns as representative of association of Sprouty and SPRED proteins to membrane vesicles. We instead reinterpret these data to confirm the fact that the Sprouty and SPRED proteins form large, multisubunit protein aggregates in living cells consistent with our biochemical studies of these proteins following over-expression and purification from bacterial cells.

Consistent with this finding, we have prepared protein lysates from a neuroblastoma cell line programmed to inducibly express the Sprouty2 protein. We cloned Sprouty cDNAs downstream of an ecdysone-responsive promoter and stably transfected the constructs into the human neuroblastoma cell line SHEP together with an expression vector encoding an ecdysone-responsive nuclear hormone receptor. Exposure of the cells to ponasterone, a synthetic mimic of ecdysone, produced a substantial induction of Sprouty2 at both the mRNA and protein levels. We also added a V5 epitope to the C-terminus of the Sprouty2 protein so it could be detected by an anti-V5 antibody.

Sprouty inducible cells were induced by ponaserone for at least 18 hours and the cells were lysed in lysis buffer containing 1% NP40. After centrifugation to remove the insoluble cell debris, the supernatant was loaded onto a Superose 6 gel filtration column and each fraction was collected. Each fraction was run on SDS gel and Sprouty2 was followed by Western blotting using V5 antibody. The results showed that Sprouty2 was detected only from early fractions eluted from the column, which corresponds to a very high molecular weight complex.

Our findings provide a second major pathway by which nitric oxide signals in the human body. The first pathway is via soluble guananyl cyclase—which uses a heme prosthetic group to sense NO, allowing NO to regulate its activity—which, in turn, regulates lots of other things including phosphodiesterases (the targets of drugs like Viagra). Accordingly, the subject compositions, including devices, provide applications to the myriad physiological targets of nitric oxide signaling. These application provide for characterization of inhibitors of nitric oxide production or nitric oxide donors for use in Sprouty or SPRED regulated pathologies. Furthermore, because Sprouty and SPRED proteins maintain stem cells in an un-differentiated state, the invention may be used to identify and characterize inhibitors of NO production, or NO donors, useful in keeping a stem cell undifferentiated, or causing it to proceed in a targeted differentiation.

The subject devices and SCRD modules preferably have predetermined redox potentials, and are electrically coupled to another component, such as redox partner, a redox modulator, an electrical conductor. The subject devices are not found in nature, and/or are isolated from its natural context; these proteins store electrons/charge and by so doing they then can be incorporated with other proteins into combinatorial biosensors/bioswitches.

The subject devices and modules may be used or incorporated as part of or in conjunction with micro- or nano-electronic or electrochemical devices, including amperometric biosensors (e.g. Zhang et al., Front Biosci. Jan. 1, 2005;10:345-52; Mehrvar et al., Anal Sci. Aug. 2004;20(8):1113-26; Albers et al., Anal Bioanal Chem. Oct. 2003;377(3):521-7; Yuqing et al. Trends Biotechnol. May 2004;22(5):227-31); DNA sensors and circuits (e.g. Drummond et al. Nat Biotechnol. Oct. 2003;21(10):1192-9; Hasty et al., Nature. Nov. 14, 2002;420(6912):224-30); cellular networks (e.g. Porod et al, Int J Neural Syst. Dec. 2003;13(6):387-95); molecular computing elements (e.g. US Pat Pub 20040235043); molecular optoelectronic devices (e.g. Wiliner et al. 1998, J Mater. Chem 8, 2543-2556); other sensors (e.g. US Patent Pub 20040245101, 20040248282, etc.), etc.

### REFERENCES

Massey, V. & Hemme rich, P. (1978) Photoreduction of flavoproteins and other biological compounds catalyzed by deazaflavins. Biochemistry 17, 9-16.
Chander, M. & Demple, B. (2004) Functional analysis of SoxR residues affecting transduction of oxidative stress signals into gene expression J. Biol. Chem. 279, 41603-41610.

Wu, J., Dunham, W. R., Weiss, B. (1995) Overproduction and physical characterization of SoxR, a [2Fe-2S] protein that governs an oxidative response regulon in Escherichia coli. J. Biol Chem. 270, 10323-7.
Drapier, J. (1997) Interplay between NO and [Fe—S] clusters: relevance to biological systems. Methods. 11, 319-29.
Ding, H. & Demple, B. (2000) Direct nitric oxide signal transduction via nitrosylation of iron-sulfur centers in the SoxR transcription activator. Proc Natl Acad Sci U S A. 97, 5146-50
Tsumura, Y., Toshima, J., Leeksma, O. C., Ohashi, K., Mizuno, K. (2005) Sprouty-4 negatively regulates cell spreading by inhibiting the kinase activity of testicular protein kinase. Biochem J. 387(Pt 3):627-37.

Engelhardt, C. M., Bundschu, K., Messerschmitt, M., Renne, T., Walter, U., Reinhard, M., Schuh, K. (2004) Expression and subcellular localization of Spred proteins in mouse and human tissues. Histochem Cell Biol. 122(6):527-38.

The foregoing description is offered by way of illustration and not by way of limitation. All publications cited in this specification, or cited by such publications, are herein incorporated by reference as if each individual publication were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method of sensing an electro/chemical signal, the method comprising the steps of:
contacting the signal with a Sprouty/SPRED protein cysteine-rich domain (SCRD) module comprising an iron:sulfur complex; and
detecting the oxidation state of the SCRD module as an indication of the signal, wherein the signal is selected from the group consisting of nitric oxide, a redox potential, and a chemical reducing agent.

2. A method of sensing redox potential in a medium, the method comprising the steps of:
contacting-the medium with a Sprouty/SPRED protein cysteine-rich domain (SCRD) module comprising an iron:sulfur complex; and
detecting the oxidation state of the SCRD module as an indication of the redox potential in the medium.

3. A method of sensing nitric oxide in a medium, the method comprising the steps of:
contacting the medium with a Sprouty/SPRED protein cysteine-rich domain (SCRD) module comprising an iron:sulfur complex; and
detecting the oxidation state of the SCRD module as an indication of nitric oxide in the medium.
